(19) United States
(12) Patent Application Publication
(10) Pub. No.: US 2020/0214936 A1
(43) Pub. Date: Jul. 9, 2020

(54) Resealing Device for Simulated Medical Fluid Bag

(71) Applicant: Pocket Nurse Enterprises, Inc., Monaca, PA (US)

(72) Inventor: Anthony J. Battaglia, Jr., Hookstown, PA (US)

(21) Appl. No.: 16/631,247

(22) PCT Filed: Jul. 16, 2018
(86) PCT No.: PCT/US2018/042281

§ 371 Date: Jan. 15, 2020

Related U.S. Application Data
(60) Provisional application No. 62/533,867, filed on Jul. 18, 2017.

(51) Int. Cl. A61J 1/14; A61J 1/10; A61J 1/16; G09B 23/28; A61M 5/162

(52) U.S. Cl. CPC A61J 1/1406; A61J 1/10; A61M 5/162; G09B 23/28; A61J 1/16

(57) ABSTRACT

A device for resealing a simulated medical fluid bag includes: a first hollow section having a first end and a second end, where the first end of the first hollow section terminates in a point; a second hollow section having a first end and a second end, where the first end of the second hollow section is connected to the second end of the first hollow section; and a fluid-tight seal disposed between the first hollow section and the second hollow section. A simulated medical fluid bag and a method of resealing a medical fluid bag are also disclosed.

### CROSS REFERENCE TO RELATED APPLICATIONS

This application claims priority to U.S. Provisional Patent Application No. 62/533,867, filed Jul. 18, 2017, the disclosure of which is hereby incorporated in its entirety by reference.

### BACKGROUND OF THE INVENTION

### Field of the Invention

The present invention relates to a device for resealing a simulated medical fluid bag, a method of resealing a medical fluid bag, and a simulated medical fluid bag including the resealing device.

### Description of Related Art

Medical personnel, including doctors, nurses, paramedics, and the like, require training to learn to place tubing (e.g., intravenous tubing or blood tubing) into a medical fluid bag (e.g., intravenous bag or blood bag) for administering contents of the bag to a patient. To place tubing in fluid communication with a medical fluid bag, the medical fluid bag is punctured.

When used in connection with patients, once punctured and used to completion, the medical fluid bag is disposed. However, in a simulated environment, where the medical fluid bag is not used or intended to be used in connection with a patient, disposal of the medical fluid bag after a single puncture by a trainee is not necessary, if the remainder of the medical fluid bag is still in useable condition. Using a new medical fluid bag for each trainee can be expensive and increase waste materials. Therefore, there is a need in the art for a device to reseal medical fluid bags used in a simulated environment. These devices are not appropriate for resealing a medical fluid bag to be subsequently used in connection with a patient.

### SUMMARY OF THE INVENTION

According to a non-limiting embodiment or aspect, provided is a device for resealing a simulated medical fluid bag including: a first hollow section having a first end and a second end, where the first end of the first hollow section terminates in a point; a second hollow section having a first end and a second end, where the first end of the second hollow section is connected to the second end of the first hollow section; and a fluid-tight seal disposed between the first hollow section and the second hollow section.

In one non-limiting embodiment or aspect, the first hollow section may include a hard plastic. The second hollow section may include a soft plastic. The second end of the second hollow section may be configured to receive a spike configured to puncture the fluid-tight seal. The first hollow section may be configured to be inserted into a tube of a medical fluid hag having a pouch so as to form a fluid-tight seal between the pouch and the device. The medical fluid bag may be a simulated intravenous bag or a simulated blood bag. A diameter of the first hollow section may be smaller than a diameter of the second hollow section. The first hollow section may define a flow hole. The first hollow section may include a ribbed region.

According to a non-limiting embodiment or aspect, provided is a simulated medical fluid bag including: a pouch for receiving medical fluid; a tube connected to the pouch; and a resealing device inserted into the tube such that a fluid-tight seal is formed between the pouch and the resealing device.

In one non-limiting embodiment or aspect, the resealing device may include: a first hollow section having a first end and a second end, where the first end of the first hollow section terminates in a point; a second hollow section having a first end and a second end, where the first end of the second hollow section is connected to the second end of the first hollow section; and a fluid-tight seal disposed between the first hollow section and the second hollow section. The pouch may be a simulated intravenous bag or a simulated blood bag. The second end of the second hollow section may be configured to receive a spike configured to puncture the fluid-tight seal. A diameter of the first hollow section may be smaller than a diameter of the second hollow section. The first hollow section may define a flow hole. The first hollow section may include a ribbed region.

According to a non-limiting embodiment or aspect, provided is a method of resealing a medical fluid bag including: providing a medical fluid hag having a pouch for receiving medical fluid and a tube connected to the pouch; puncturing the pouch by inserting a spike into the tube; and inserting a resealing device into the tube such that a fluid-tight seal is formed between the pouch and the resealing device.

In one non-limiting embodiment or aspect, the resealing device may include: a first hollow section having a first end and a second end, where the first end of the first hollow section terminates in a point; a second hollow section having a first end and a second end, where the first end of the second hollow section is connected to the second end of the first hollow section; and a fluid-tight seal disposed between the first hollow section and the second hollow section. The pouch may be a simulated intravenous bag or a simulated blood bag. The second end of the second hollow section may be configured to receive a second spike configured to puncture the fluid-tight seal.

Further embodiments or aspects are set forth in the following numbered clauses:

Clause 1: A device for resealing a simulated medical fluid bag comprising: a first hollow section comprising a first end and a second end, wherein the first end of the first hollow section terminates in a point; a second hollow section comprising a first end and a second end, wherein the first end of the second hollow section is connected to the second end of the first hollow section; and a fluid-tight seal disposed between the first hollow section and the second hollow section.

Clause 2: The device of clause 1, wherein the first hollow section comprises a hard plastic.

Clause 3: The device of clause 1 or 2, wherein the second hollow section comprises a soft plastic.

Clause 4: The device of any of clauses 1-3, wherein the second end of h second hollow section is configured to receive a spike configured to puncture the fluid-tight seal.

Clause 5: The device of any of clauses 1-4, wherein the first hollow section is configured to be inserted into a tube of a medical fluid bag comprising a pouch so as to form a fluid-tight seal between the pouch and the device.

Clause 6: The device of clause 5, wherein the medical id bag is a simulated intravenous bag or a simulated blood bag.

Clause 7: The device of any of clauses 1-6, wherein a diameter of the first hollow section is smaller than a diameter of the second hollow section.

Clause 8: The device of any of clauses 1-7, wherein the first hollow section defines a flow hole.

Clause 9: The device of any of clauses 1-8 wherein the first hollow section comprises a ribbed region.

Clause 10: A simulated medical fluid bag comprising: a pouch for receiving medical fluid; a tube connected to the pouch; and a resealing device inserted into the tube such that a fluid-tight seal is formed between the pouch and the resealing device.

Clause 11: The simulated medical fluid bag of clause 10, wherein the resealing device comprises: a first hollow section comprising a first end and a second end, wherein the first end of the first hollow section terminates in a point; a second hollow section comprising a first end and a second end, wherein the first end of the second hollow section is connected to the second end of the first hollow section; and a fluid-tight seal disposed between the first hollow section and the second hollow section.

Clause 12: The simulated medical fluid bag of clause 10 or 11, wherein the pouch is a simulated intravenous bag or a simulated blood bag.

Clause 13: The simulated medical fluid bag of clause 11, wherein the second end of the second hollow section is configured to receive a spike configured to puncture the fluid-tight seal.

Clause 14: The simulated medical fluid bag of any of clauses 11-13, wherein a diameter of the first hollow section is smaller than a diameter of the second hollow section.

Clause 15: The simulated medical fluid bag of any of clauses 11-14, wherein the first hollow section defines a flow hole.

Clause 16: The simulated medical fluid bag of any of clauses 11-15, wherein the first hollow section comprises a ribbed region.

Clause 17: A method of resealing a medical fluid bag comprising: providing a medical fluid bag comprising a pouch for receiving medical fluid and a tube connected to the pouch; puncturing the pouch by inserting a spike into the tube; and inserting a resealing device into the tube such that a fluid-tight seal is formed between the pouch and the resealing device.

Clause 18: The method of clause 17, wherein the resealing device comprises: a first hollow section comprising a first end and a second end, wherein the first end of the first hollow section terminates in a point; a second hollow section comprising a first end and a second end, wherein the first end of the second hollow section is connected to the second end of the first hollow section; and a fluid-tight seal disposed between the first hollow section and the second hollow section.

Clause 19: The method of clause 17 or 18, wherein the pouch is a simulated intravenous bag or a simulated blood bag.

Clause 20: The method of clause 18, wherein the second end of the second hollow section is configured to receive a second spike configured to puncture the fluid-tight seal.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a,” “an,” and “the” include plural referents unless the context clearly dictates otherwise.

### BRIEF DESCRIPTION OF THE DRAWINGS

FIG. 1A shows an embodiment of a resealing device according to the present invention;

FIG. 1B shows an embodiment of another resealing device according to the present invention;

FIG. 1C shows an embodiment of another resealing device according to the present invention;

FIG. 1D shows an embodiment of another resealing device according to the present invention;

FIG. 1E shows an embodiment of another sealing device according to the present invention;

FIG. 2A shows a medical fluid bag containing intravenous fluid;

FIG. 2B shows a medical fluid bag containing blood;

FIG. 3A shows a punctured medical fluid bag;

FIG. 3B shows a simulated medical bag including a resealing device according to the present invention; and

FIG. 4 shows an embodiment of another resealing device according to the present invention.

### DESCRIPTION OF THE INVENTION

The present invention will now be described with reference to the accompanying figures. For purposes of the description hereinafter, the terms “upper” “lower”, “right”, “left”, “vertical”, “horizontal”, “top”, “bottom”, and derivatives thereof shall relate to the invention as it is oriented in the drawing figures. However, it is to be understood that the invention may assume various alternative variations and step sequences, except where expressly specified to the contrary. It is to be understood that the specific system illustrated in the attached figures and described in the following specification is simply an exemplary embodiment of the present invention. Hence, specific dimensions and other physical characteristics related to the embodiments disclosed herein are not to be considered as limiting.

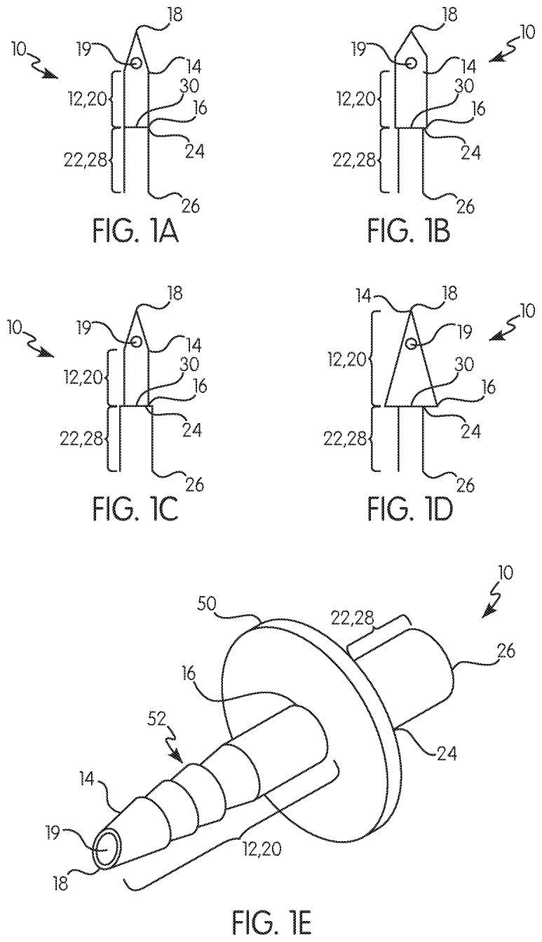

Referring to FIGS. 1A-1E, non-limiting embodiments of a resealing device 10 for resealing a simulated medical fluid bag are shown. The resealing device 10 may include a first hollow section 12. The first hollow section 12 may be a hollow tube (FIGS. 1A-1C, 1E), or the first hollow section may be another hollow shape (FIG. 1D). As shown in FIG. 1D, the first hollow section 12 may be a hollow triangular section, tapered along its length so as to plug a pierced hole in a simulated medical fluid bag when the bag is punctured by the first hollow section 12. The first hollow section 12 may include a first end 14 and a second end 16. The first hollow section 12 may have a circular cross-sectioned second end 16 regardless of its overall shape, so that the first hollow section 12 includes a diameter. At the first end 14, the first hollow section 12 may include a point 18. The point 18 may be a single point, or any other sharp end, sharp enough to puncture a medical fluid bag. The point 18 may be capable of puncturing plastic. However, as shown in FIG. 1E, the point 18 need not necessarily be a sharp point. The first hollow section 12 may also define a flow hole 19. The first hollow section 12 may include a hard plastic 20. A portion or the entire first hollow section 12 may be made of the hard plastic 20. The hard plastic 20 may be any material hard enough to puncture a simulated medical fluid bag.

With continued reference to FIGS. 1A-1E, the flow hole 19 may be positioned anywhere along the first hollow section 12. In some non-limiting embodiments, the flow hole 19 may be defined along the first hollow section 12 closer to the first end 14 than the second end 16; however, the opposite arrangement is contemplated. In some embodiments, the flow hole 19 may be defined proximate to the point 18 in a tapered section of the first hollow section 12. In some embodiments, the flow hole 19 may be defined at the point 18 (see FIG. 1E). The flow hole 19 may be configured to allow fluid to flow therethrough and into the first hollow section 12.

With continued reference to FIGS. 1A-1E, the resealing device 10 may include a second hollow section 22. The second hollow section 22 may be a hollow tube or any other hollow shape. The second hollow section 22 may include a first end 24 and a second end 26. In some embodiments, the hollow section 22 may take the form of tubing. The second end 26 may be configured to receive a spike in fluid communication with an intravenous tube. The second hollow section 22 may include a soft plastic 28 (e.g., having a hardness value lower than the hard plastic 20). A portion or the entire second hollow section 22 may be made of the soft plastic 28. The soft plastic 28 may be the same material as a tube in a medical fluid bag.

With continued reference to FIGS. 1A-1E, the first end 24 of the second hollow section 22 may be connected to the second end 16 of the first hollow section 12, such that the first hollow section 12 is in fluid communication with the second hollow section 22 (without the below-described seal disposed therebetween). In some non-limiting embodiments (see FIG. 1A), a diameter of the first hollow section 12 is identical to a diameter of the second hollow section 22. The first hollow section 12 and the second hollow section 22 may be the same diameter along the entire length of the hollow sections 12, 22. The first hollow section 12 and the second hollow section 22 may be co-axial with one another. In some non-limiting embodiments (see FIGS. 1B and 1D), a diameter of the first hollow section 12 is larger than a diameter of the second hollow section 22. The first hollow section 12 may be of a larger diameter than the second hollow section 22 along the entire length of the hollow sections 12, 22 excluding the tapered section constituting the point 18 of the first hollow section 12 or may be of a larger diameter than the second hollow section 22 along only a portion of the length. The first hollow section 12 and the second hollow section 22 may be co-axial with one another. In some non-limiting embodiments (see FIGS. 1C, 1E), a diameter of the first hollow section 12 is smaller than a diameter of the second hollow section 22. The first hollow section 12 may be of a smaller diameter than the second hollow section 22 along the entire length of the hollow sections 12, 22. The first hollow section 12 and the second hollow section 22 may be co-axial with one another. The different diameters of the first hollow section 12 and the second hollow section 22 (see FIGS. 1B, 1C, 1D, and 1E) may help seal a punctured pouch of a medical fluid bag so that there is a fluid-tight seal formed, such as when the bag or bag opening is stretched due to the bag being previously punctured or having a device according to the present disclosure inserted therein. The first hollow section 12 may be tapered along its length (see FIGS. 1D, 1E) to help seal a punctured pouch of a medical fluid bag so that there is a fluid-tight seal formed.

With continued reference to FIGS. 1A-1E, the resealing device 10 may further include a seal 30. The seal 30 may be disposed between the first hollow section 12 and the second hollow section 22. The seal 30 may also be disposed in the second hollow section 22. The seal 30 may be a fluid-tight seal to prevent fluid, such as intravenous fluid or blood, from flowing from the first hollow section 12 to the second hollow section 22. The seal 30 may be made of a material puncturerable by a spike made of hard plastic in fluid communication with a tube. The seal 30 may be made of the same material as a medical fluid bag.

Referring to FIG. 1E, the resealing device 10 may further include a disc 50 positioned between the first hollow section 12 and the second hollow section 22. The disc 50 may be co-axial with the seal 30 (not shown), which may be positioned in an opening of the disc 50. The disc 50 may help the resealing device 10 further form a fluid-tight seal. The disc may also aid the user to insert the resealing device 10 into a medical fluid bag and/or to insert a spike into the resealing device 10 (e.g., the second hollow section 22 thereof). It will be appreciated that the disc 50 is not limited to the circular disc pictured in FIG. 1E, but the disc may be other shapes as well, such as rectangular, oblong, triangular, and the like.

With continued reference to FIG. 1E, the first hollow section 12 of the resealing device 10 may include a ribbed region 52 along its outside. The ribbed region 52 may run along the entire length of the first hollow section 12 or along only a portion of the length of the first hollow section 12. Each rib of the ribbed region 52 may be frustoconical in shape, such that the larger diameter of the frustoconical shape is closer to the second end 16 of the first hollow section 12 compared to the smaller diameter of the frustoconical shape. The ribbed region 52 may help secure the resealing device 10 in medical fluid bag by the ribs making it more difficult to pull the resealing device 10 back out of the medical fluid bag.

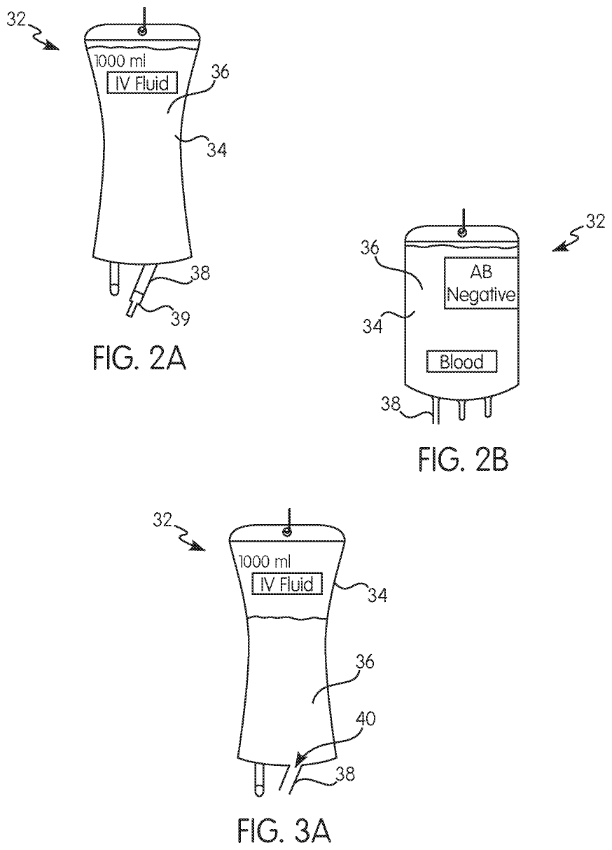

Referring to FIGS. 2A and 2B, medical fluid bags 32, which are capable of being resealed using the resealing device 10, are shown. The medical fluid bag 32 may be any bag used to hold medical fluid. The medical fluid bag 32 may be a blood bag (FIG. 2B) containing blood or an intravenous bag (FIG. 2A) holding other intravenous fluid. The medical fluid bag 32 may also be a simulated medical fluid bag that holds simulated medical fluid. The medical fluid bag 32 may include a pouch 34, and the pouch 34 may be configured for holding a fluid 36, such as medical fluid or simulated medical fluid. The medical fluid bag 32 may also include a tube 38 connected to the pouch 34. A cap 39 may be positioned at a distal end of the tube 38 relative to the pouch 34 to cap the tube 38 so that no fluid 36 can exit the distal end of the tube 38 and no spike can be inserted into the tube 38 until the cap 39 is removed. It will of course be appreciated that if a medical bag 32 includes actual medical fluid, the medical bag 32 is not used or intended to be used with a patient after the medical bag 32 has been punctured a first time and/or when subsequently re-sealed to form a simulated medical bag.

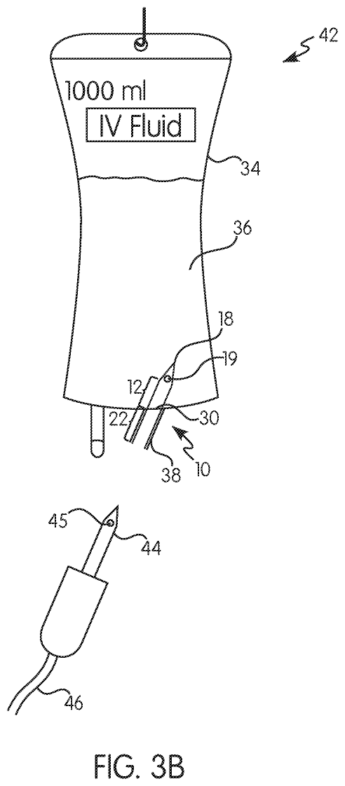

Referring to FIG. 3A, the tube 38 and the pouch 34 may be separated by the material of the pouch 34 until such material is punctured to create a puncture hole 40, rendering the tube 38 in fluid communication with the pouch 34. The puncture hole 40 may be formed by inserting a spike in the tube 38 to puncture the pouch 34, and the spike may be in fluid communication with another tube, such as an intravenous tube, to flow the fluid 36 from the pouch 34 to, for example, a patient. Fluid 36 from the pouch 34 may flow through the puncture hole 40 and through the tube 38 to a patient, for example, after the pouch 34 is punctured. Fluid 36 flowing out of the pouch 34 through the tube 38 may lower a fluid level in the pouch 34.

Referring to FIG. 3B, a simulated medical fluid bag 42 is shown. The simulated medical fluid bag 42 may be identical to the medical fluid bag 32 previously described, except the simulated medical fluid bag 42 may further include the resealing device 10. The resealing device 10 may be inserted into the tube 38. Inserting the resealing device 10 into the tube 38 may form a fluid-tight seal between the pouch 34 and the resealing device 10 such that the fluid 36 can no longer flow out of the pouch 34 through the puncture hole 40 after the resealing device 10 is inserted. The seal 30 of the resealing device 10 may provide the fluid-tight seal for the simulated medical fluid bag 42. Further, the resealing device 10 may be inserted into the tube 38 so as to be positioned to form a seal so that the fluid 36 from the pouch 34 cannot flow around the outside of the first hollow section 12 or second hollow section 14 of the resealing device 10 where the first and second hollow sections 12, 22 contact the pouch 34. The relative diameters of the first hollow section 12 and the second hollow section 22 may aid in sealing this region of the pouch 34. In other words, the resealing device 10 being inserted into the tube 38 may reseal the pouch 34, preventing the fluid 36 from flowing out the previously punctured puncture hole 40.

With continued reference to FIG. 3B, a spike 44 may be inserted into the tube (also the second end 26 of the second hollow section 22) of the simulated medical fluid bag 42 including the resealing device 10, such that the same pouch 34 of the medical fluid bag 32 (e.g., from FIG. 3A) may be punctured multiple times. This allows for cost savings when training medical personnel to place tubing (e.g., intravenous tubing or blood tubing) into a medical fluid bag. The spike 44 may be hollow and may define a hole 45 through which the fluid 36 from the pouch 34 may flow when the spike 44 is inserted into the pouch 34 through the tube 38 after puncturing the seal 30 of the resealing device 10. The spike 44 may be in fluid communication with a tube 46, such as an intravenous tube, so that the fluid 36 from the pouch 34 can flow through the spike 44 and into the tube 46. After the spike 44 punctures the seal 30 of the resealing device 10 and is eventually removed from the pouch 34, further resealing devices 10 may be inserted into the tube 38 of the simulated medical fluid bag 42 so that the pouch 34 may be punctured again for training purposes. In some non-limiting embodiments, the same pouch 34 may be punctured by a spike 44 twice (once to the original fluid bag 32 and once to the fluid bag 42 after the resealing device 10 has been inserted). However, in other non-limiting embodiments, the same pouch 34 may be punctured more than twice, with another resealing device 10 being inserted into the tube 38 between each puncture. This may be done by removing the previously inserted resealing device 10 and replacing it with another resealing device 10 or by inserting another resealing device 10 within the previously inserted resealing device 10. In this way, a new pouch 34 need not be used for each puncture made by a trainee, saving materials and costs associated with training. It will be appreciated that due to sanitary concerns, regulations, and other concerns, the resealing device 10 would not be used to reuse medical fluid bags used on actual patients. In use, in some embodiments, the simulated medical bag will be kept upside down when removing and after removal of the initial spike of the hag and during insertion of the resealing device 10 to prevent leakage of the fluid through the unsealed opening.

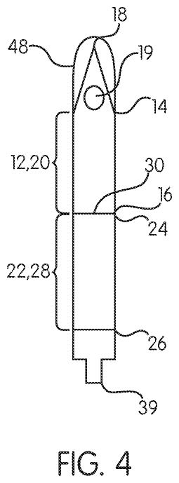

Referring to FIG. 4, another non-limiting embodiment of the resealing device 10 including further features is shown. The resealing device 10 may include a protective cover 48 over the point 18 of the resealing device 10. The protective cover 48 may protect a trainee or other user from being poked by the point 18. The protective cover 48 may be removed by the trainee or instructor prior to inserting the resealing device 10 into the tube 38. The resealing device may also include the protective cap 39 used to cap the distal end of the tube 38 in the original medical fluid bag 32. The protective cap 39 may be positioned on the second end 26 of the second hollow section 22. The protective cap 39 may be removed by the trainee prior to inserting a spike 44 into the second end 26 of the second hollow section 22. After removing the protective cover 48, the resealing device 10 may then be inserted into the bag 32 or 42, such as explained above, while upside down to prevent fluid leakage/spills. The resealed bag could then be hung, for example, as shown in FIG. 3B, such as on an IV pole for use by the next trainee/student who would remove the cap 39 and proceed to spike the bag.

Although the invention has been described in detail for the purpose of illustration based on what is currently considered to be the most practical and preferred embodiments, it to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A device for resealing a simulated medical fluid bag comprising:
a first hollow section comprising a first end and a second end, wherein the first end of first hollow section terminates in a point;
a second hollow section comprising a first end and a second end, wherein the first end of the second hollow section is connected to the second end of the first hollow section; and
a fluid-tight seal disposed between the first hollow section and the second hollow section.

2. The device of claim 1, wherein the first hollow section comprises a hard plastic.

3. The device of claim 1, wherein the second hollow section comprises a soft plastic.

4. The device of claim 1, wherein the second end of the second hollow section is configured to receive a spike configured to puncture the fluid-tight seal.

5. The device claim 1, wherein the first hollow section is configured to be inserted into a tube of a medical fluid bag comprising a pouch so as to form a fluid-tight seal between the pouch and the device.

6. The device of claim 5, wherein the medical fluid bag is a simulated intravenous bag or a simulated blood bag.

7. The device of claim 1, wherein a diameter of the first hollow section is smaller than a diameter of the second hollow section.

8. The device of claim 1, wherein the first hollow section defines a flow hole.

9. The device of claim 1, wherein the first hollow section comprises a ribbed region.

10. A simulated medical fluid bag comprising:
a pouch for receiving medical fluid;
a tube connected to the pouch; and
a resealing device inserted into the tube such that a fluid-tight seal is formed between the pouch and the resealing device.

11. The simulated medical fluid bag of claim 10, wherein the resealing device comprises:
a first hollow section comprising a first end and a second end, wherein the first end of the first hollow section terminates in a point;
a second hollow section comprising a first end and a second end, wherein the first end of the second hollow section is connected to the second end of the first hollow section; and
a fluid-tight seal disposed between the first hollow section and the second hollow section.

12. The simulated medical fluid bag of claim 10, wherein the pouch is a simulated intravenous bag or a simulated blood bag.

13. The simulated medical fluid bag of claim 11, wherein the second end of the second hollow section is configured to receive a spike configured to puncture the fluid-tight seal.

14. The simulated medical fluid bag of claim 11, wherein a diameter of the first hollow section is smaller than a diameter of the second hollow section.

15. The simulated medical fluid bag of claim 11, wherein the first hollow section defines a flow hole.

16. The simulated medical fluid bag of claim 11, wherein the first hollow section comprises a ribbed region.

17. A method of resealing a medical fluid bag comprising:
providing a medical fluid bag comprising a pouch for receiving medical fluid and a tube connected to the pouch;
puncturing the pouch by inserting a spike into the tube; and
inserting a resealing device into the tube such that a fluid-tight seal is formed between the pouch and the resealing device.

18. The method of claim 17, wherein the resealing device comprises:
a first hollow section comprising a first end and a second end, wherein the first end of the first hollow section terminates in a point;
a second hollow section comprising a first end and a second end, wherein the first end of the second hollow section is connected to the second end of the first hollow section; and
a fluid-tight seal disposed between the first hollow section and the second hollow section.

19. The method of claim 17, wherein the pouch is a simulated intravenous bag or a simulated blood bag.

20. The method of claim 18, wherein the second end of the second hollow section is configured to receive a second spike configured to puncture the fluid-tight seal.
